(19) United States
(12) Patent Application Publication
(10) Pub. No.: US 2007/0238883 A1
(43) Pub. Date: Oct. 11, 2007

(54) Process for the preparation of(s)-(+)-N,N-dimethyl-3-(1-Naphthalenyloxy)-3-(2-Thienyl)propanamine, A duloxetine intermediate

(76) Inventors: Santiago Ini, Haifa (IL); Yaron Shmuely, Hedera (IL); Mili Abramov, Givataim (IL)

Correspondence Address: KENYON & KENYON LLP, ONE BROADWAY, NEW YORK, NY, 10004, US

(21) Appl. No.: 11/706,009

(22) Filed: Feb. 13, 2007

Related U.S. Application Data
(60) Provisional application No. 60/773,065, filed on Feb. 13, 2006.
(60) Provisional application No. 60/786,488, filed on Mar. 27, 2006.
(60) Provisional application No. 60/789,380, filed on Apr. 4, 2006.
(60) Provisional application No. 60/791,102, filed on Apr. 10, 2006.
(60) Provisional application No. 60/815,167, filed on Jun. 19, 2006.

(51) Int. Cl. C07D 333/16

(52) U.S. Cl. 549/78

(57) ABSTRACT

Provided is a process for preparing a duloxetine intermediate, (S)-(+)-N,N-Dimethyl-3-(1-naphthalenyloxy)-3-(2-thienyl)propanamine (DNT), and its conversion to duloxetine or a pharmaceutically acceptable salt thereof.

### CROSS-REFERENCE TO RELATED APPLICATIONS

The present application claims the benefit of the following U.S. Provisional Patent Nos. 60/773,065 filed 13 Feb. 2006, 60/786,488 filed 27 Mar. 2006, 60/789,380 filed 4 Apr. 2006, 60/791,102 filed 10 Apr. 2006 and 60/815,167 filed 19 Jun. 2006, hereby incorporated by reference.

### FIELD OF THE INVENTION

The present invention provides processes for preparing a duloxetine intermediate. The present invention also provides processes for converting the duloxetine intermediate into duloxetine HCl.

### BACKGROUND OF THE INVENTION

Duloxetine is a dual reuptake inhibitor of the neurotransmitters serotonin and norepinephrine. It has application for the treatment of stress urinary incontinence (SUI), depression, and pain management. Duloxetine hydrochloride has the following chemical name (+)-N-methyl-3-(1-naphthalenyloxy)-3-(2-thienyl)propanamine hydrochloric acid salt and structure:

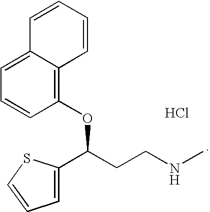

Duloxetine base, as well as processes for its preparation, is disclosed in U.S. Pat. No. 5,023,269 (U.S. '269). EP Patent No. 457559, and U.S. Pat. No. 5,491,243 (U.S. '243) and U.S. Pat. No. 6,541,668 provide an improved synthetic route for the preparation of duloxetine base. U.S. '269 describes the preparation of duloxetine base by reacting (S)-(−)-N,N-Dimethyl-3-(2-thienyl)-3-hydroxypropanamine and fluoronaphthalene with sodium hydride in DMA (Stage a), followed by demethylation with Phenyl chloroformate or trichloroethyl chloroformate (Stage b) and basic hydrolysis (Stage c) according the following scheme:

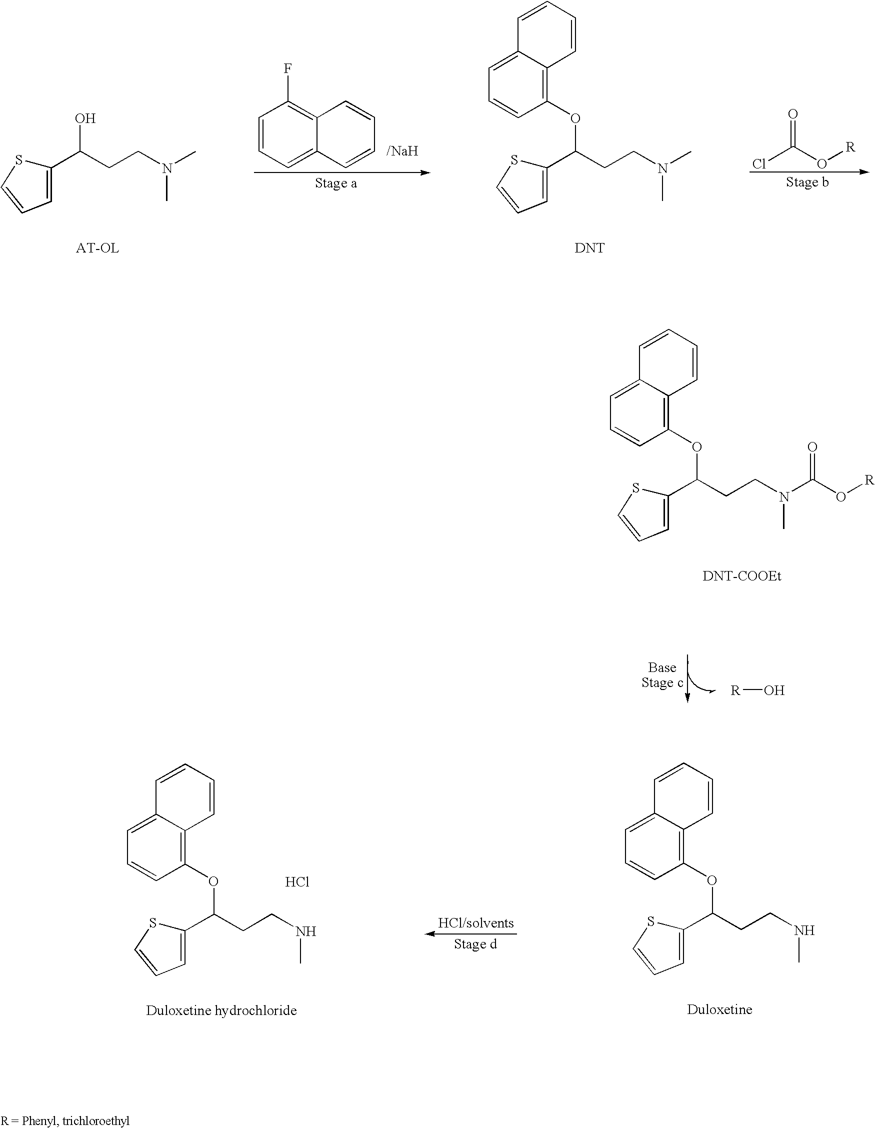

The conversion of duloxetine base to its hydrochloride salt is described in U.S. Pat. No. 5,491,243 and in Wheeler W. J., et al, J. Label.Cpds.Radiopharm, 1995, 36, 312. In both cases the reactions are performed in ethyl acetate.

In U.S. Pat. No. 5,362,886, the process described in Stage a) is performed in the presence of Potassium salts, such as potassium benzoate or potassium acetate in the presence of sodium hydride. In U.S. '668, the process described in Stage a) is performed in the presence of potassium tert-butoxide and 1,3 dimethyl-2-imidazolidinone or N-methylpyrrolidine, at 110° C. In WO Publication No. 04/056795 this stage is performed in the presence of phase transfer catalyst and a base in DMSO (examples 1 and 4).

The drawbacks of the processes described in the above patents and publication include the use of sodium hydride, which requires special handling and safety conditions, as sodium hydride reacts violently with water, liberating and igniting hydrogen, and the use of 1,3-dimethyl-2-imidazolidinone, N-methylpyrrolidine or a phase transfer catalyst, which results in high cost.

Cost effective methods of synthesizing duloxetine intermediates and duloxetine HCl, utilizing safe reagents are highly desirable.

### SUMMARY OF THE INVENTION

In one embodiment, the present invention provides a process for preparing (S)-(+)-N,N-Dimethyl-3-(1-naphthalenyloxy)-3-(2-thienyl)propanamine (DNT), comprising combining S-(−)-N,N-Dimethyl-3-Hydroxy-3-(2-Thienyl)Propanamine (AT-OL) with a base selected from the group consisting of: alkali metal hydroxide, sodium metal alkoxides, lithium metal alkoxides, and a naphthalene selected from the group consisting of 1-fluoronaphthalene, 1-chloronaphthalene and mixtures thereof in a polar aprotic solvent selected from the group consisting of: C5-C8 aromatic hydrocarbons, ionic liquid, dimethyl Sulfoxide (DMSO), dimethylformamide (DMF), dimethylacetamide (DMA), acetonitrile, sulfolane, nitromethane, propylene carbonate and mixtures thereof to obtain DNT, wherein the reaction is conducted in the absence of a phase transfer catalyst.

Also provided is a process for preparing duloxetine or a pharmaceutically acceptable salt thereof, comprising preparing DNT or salts thereof according to the above process and converting the DNT to duloxetine or a pharmaceutically acceptable salt.

### DETAILED DESCRIPTION OF THE INVENTION

As used herein the term “AT-OL” refers to: (S)-(−)-N,N-Dimethyl-3-(2-thienyl)-3-hydroxypropanamine.

As used herein the term “DNT” refers to: (S)-(+)-N,N-Dimethyl-3-(1-naphthalenyloxy)-3-(2-thienyl)propanamine.

The present invention provides a process for preparing DNT or salts thereof without the use of a hydride base or a phase transfer catalyst. The process of the present invention is suitable for use on industrial scale.

In one embodiment, DNT is prepared by combining AT-OL, a base, specifically, alkali metal hydroxide, sodium metal alkoxides, lithium metal alkoxides, and 1-fluoronaphthalene or 1-chloronaphthalene, and a polar aprotic solvent selected from the group consisting of: C₅-C₈ aromatic hydrocarbons, ionic liquid, dimethyl Sulfoxide (DMSO), dimethylformamide (DMF), dimethylacetamide (DMA), acetonitrile, sulfolane, nitromethane and propylene carbonate. This embodiment is carried out in the absence of a phase transfer catalyst.

Preferably, AT-OL is dissolved in the polar aprotic solvent, and the solution is then combined with a base which combination is further combined with 1-fluoronaphthalene or 1-chloronaphthalene to obtain a reaction mixture.

Preferably, the base is potassium hydroxide (KOH), sodium methoxide, or sodium hydroxide (NaOH). The base may be added portion wise in order to increase the chemical yield.

The C₅-C₈ aromatic hydrocarbons may be selected from the group consisting of toluene and xylene. The ionic liquid may be selected from the group consisting of alkylammonium halides, alkylphosphonium halides, N-alkylpyridinium halides, N-N-dialkylimidazolium halides, tetraalkylammonium tetraalkylborides, 1-alkyl-3-methylimidazolium trifluoromethanesulfonate salts, monoalkylammonium nitrate salts, halogeoaluminate, chlorocuprate and 1-butyl-3-methylimidazolium tetrafluoroborate. More preferably, the ionic liquid is 1-butyl-3-methylimidazolium tetrafluoroborate Most preferably, the polar aprotic solvent is DMA or DMSO. As used herein the term “ionic liquid” refers to salts whose melting point is relatively low (below about 100° C.). In particular, the salts that are liquid at room temperature and are called room temperature ionic liquids, or RTILs.

The reagents can be used in different ratios. Preferably, the AT-OL is used in at least 1:1 molar ratio to the solvent used, base used or naphthalene used. In one embodiment, the ratio of AT-OL to solvent is about 1 g to about 6 ml; AT-OL to base is about 1 to about 1 by mol equivalent; and/or ratio of AT-OL to naphthalene is about 1 to about 2 by mol equivalent.

In one embodiment, after addition of naphthalene, the reaction mixture is heated to a temperature of from about room temperature to about the reflux temperature of the solvent. Preferably, after heating, the mixture is maintained, while stirring, for about 20 minutes to about 5 days. The reaction mixture may be maintained even in the absence of heating.

The product prepared by the above process can be obtained in high enantiomeric excess. Preferably, the amount of the R enantiomer is less than about 15% as measured by area percentage HPLC, more preferably less than about 10%, and most preferably about 0.5%.

The DNT prepared according to the above process may be recovered. In one embodiment, water and a water immiscible organic solvent such as ethyl acetate are added to the reaction mixture to obtain two phases. The phases are then separated and the organic phase is concentrated to obtain a dry residue. Prior to separation, the DNT may be washed in order to remove inorganic impurities, or organic impurities that are miscible in water. An acid such as HCl may also be added to the reaction mixture to quench the reaction.

The DNT obtained can be converted to a salt. Such salts can be prepared by reacting DNT with an organic or inorganic acid. Examples of organic acids include maleic, succinic, fumaric citric, acetic, oxalic and benzensulfonic acids. Examples of inorganic acids include phosphoric, hydrochloride, hydrobromide, hydroiodide, sulfuric and nitric acids.

The DNT or salts thereof prepared according to the above process may be recovered by any method known in the art, such as separating the phases, and concentrating the organic phase until a dry residue is formed or as an acid salt. Prior to separation, the DNT may be washed in order to remove inorganic impurities, or organic impurities that are miscible in water.

In another embodiment, the present invention provides processes for converting the obtained DNT to duloxetine, or a pharmaceutically acceptable salt thereof such as duloxetine hydrochloride.

The conversion of DNT to a pharmaceutically acceptable salt of duloxetine may be performed by any method known in the art, such as the one described in U.S. Pat. No. 5,023,269 or US20060194869 for making duloxetine HCl. The disclosure of these applications for conversion of DNT to duloxetine HCl is incorporated herein by reference. Preferably, the conversion is performed by dissolving DNT in an organic solvent, and combining it with an alkyl haloformate. That step will yield duloxetine alkyl carbamate, which can be combined with an organic solvent and a base, to yield duloxetine. The duloxetine may then be converted to a pharmaceutically acceptable salt. More preferably, the conversion is performed by dissolving DNT in a water immiscible organic solvent; adding alkyl chloroformate at a temperature of about 5° C. to less than about 80° C. to obtain duloxetine alkyl carbamate, combining the duloxetine alkyl carbamate with an organic solvent and a base; maintaining the reaction mixture at reflux temperatures for at least 1 to 3 hours; cooling, and adding water and an additional amount of an organic solvent; recovering duloxetine; combining the duloxetine with a solvent; adding hydrochloric acid until a pH of about 3 to about 4 is obtained; maintaining the reaction mixture to obtain a solid residue; and recovering duloxetine HCl.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

HPLC method for measuring enantiomeric purity of DNT:

| | |
|---|---|
| Column: | Daicel Chiralcel OD, 10 μm, 250 × 4.6 mm |
| Eluent: | 970 mL Hexane; 30 mL Isopropanol; 2 mL |
| | Diethylamine |
| Sample volume: | 100 μL |
| Flow: | 0.8 mL/min |
| Detector: | 230 nm |
| Column temperature: | 30° C. |
| Sample concentration: | 0.02 mg/mL |

HPLC method for measuring enantiomeric purity of duloxetine:

| | |
|---|---|
| Column: | Daicel Chiralcel OD, 10 μm, 250 × 4.6 mm |
| Eluent: | 900 mL Hexane; 100 mL Isopropanol; 2 mL |
| | Diethylamine |
| Sample volume: | 100 μL |
| Flow: | 1.0 mL/min |
| Detector: | 230 nm |
| Column temperature: | 20° C. |
| Sample concentration: | 0.5 mg/mL |

### Example 1

A 150 ml reactor three necked flask equipped with mechanical stirrer, thermometer, and condenser was charged with 10 g of AT-OL and 60 ml DMSO at room temperature. The mixture was stirred until complete dissolution, and 7.11 g of KOH were added and stirred for an additional time. After 15 minutes, 8 ml of 1-fluoronaphthalene were added, and the solution was heated to 60° C., and stirred for 20 hours.

To the reaction mixture was added water, followed by 10 ml HCl (5%) and 60 ml ethyl acetate. After phase separation, the organic phase was washed with brine, and concentrated to dryness to give 18.14 g of brownish oil containing 10.57% enantiomer R.

### Example 2

A 150 ml reactor three necked flask equipped with mechanical stirrer, thermometer, and condenser was charged with 10 g of AT-OL and 60 ml DMSO at 20° C. The mixture was stirred until complete dissolution, and 4.20 g of NaOH were added and stirred for an additional time. After 15 minutes, 8 ml of 1-fluoronaphthalene were added, the solution was heated to 60° C., and stirred for 5 days or till full consumption of AT-OL.

To the reaction mixture was added water, followed by 5 ml AcOH and 60 ml ethyl acetate. After phase separation, the water phase was extracted with ethyl acetate and the organic extracts were combined, and concentrated to dryness to give 17.34 g of brownish oil containing 8.80% enantiomer R.

### Example 3

A 100 ml reactor three necked flask equipped with mechanical stirrer, thermometer, and condenser was charged with 10 g of AT-OL and 60 ml DMSO at room temperature under N₂. The mixture was stirred until complete dissolution, and 7.11 g of KOH were added and stirred for an additional time. After 15 minutes, 8 ml of 1-fluoronaphthalene were added, the solution was heated to 40° C., and stirred for 120 hours (or until completion).

To the reaction mixture was added water, followed by 10 ml HCl (5%) and 60 ml ethyl acetate. After phase separation, the organic phase was washed with brine, and concentrated to dryness to give brownish oil containing 5.80% enantiomer R.

### Example 4

A 250 ml two necked flask equipped with magnetic stirrer, and condenser was charged with 10 g of AT-OL and 60 ml ACN at room temperature under N₂. The mixture was stirred until complete dissolution, and 7.11 g of KOH were added and stirred for an additional time. After 15 minutes, 8 ml of 1-fluoronaphthalene were added, the solution was heated to 60° C., and stirred for 27 hours.

To the reaction mixture was added water, followed by 10 ml HCl (5%) and 60 ml ethyl acetate. After phase separation, the organic phase was washed with brine, and concentrated to dryness to give 22.2 g of brownish oil containing 0.53% enantiomer R.

### Example 5

A 250 ml two necked flask equipped with magnetic stirrer, and condenser was charged with 10 g of AT-OL and 60 ml DMF at room temperature under N₂. The mixture was stirred until complete dissolution, and 7.11 g of KOH were added and stirred for an additional time. After 15 minutes, 8 ml of 1-fluoronaphthalene were added, the solution was heated to 60° C., and stirred for 27 hours.

To the reaction mixture was added water, followed by 10 ml HCl (5%) and 60 ml ethyl acetate. After phase separation, the organic phase was washed with brine, and concentrated to dryness to give 16.16 g of brownish oil containing 1.49% enantiomer R.

### Example 6

A 250 ml two necked flask equipped with magnetic stirrer, and condenser was charged with 10 g of AT-OL and 60 ml DMA at room temperature under N₂. The mixture was stirred until complete dissolution, and 7.11 g of KOH were added and stirred for an additional time. After 15 minutes, 8 ml of 1-fluoronaphthalene were added, the solution was heated to 60° C., and stirred for 27 hours.

To the reaction mixture was added water, followed by 10 ml HCl (5%) and 60 ml ethyl acetate. After phase separation, the organic phase was washed with brine, and concentrated to dryness to give 20.37 g of brownish oil containing 1.35% enantiomer R.

### Example 7

A 100 ml reactor three necked flask equipped with mechanical stirrer, thermometer, and condenser was charged with 10 g of AT-OL and 60 ml DMSO at room temperature under N₂. The mixture was stirred until complete dissolution, and 7 g of Na⁺MeO⁻ were added and stirred for an additional time. After 15 minutes, 8 ml of 1-fluoronaphthalene were added, the solution was heated to 60° C., and stirred for 26 hours.

To the reaction mixture was added water, followed by 10 ml HCl (5%) and 60 ml ethyl acetate. After phase separation, the organic phase was washed with brine, and concentrated to dryness to give 19 g of brownish oil containing 5.87% enantiomer R.

### Example 8

A 100 ml reactor three necked flask equipped with mechanical stirrer, thermometer, and condenser was charged with 10 g of AT-OL and 60 ml DMSO at room temperature under N₂. The mixture was stirred until complete dissolution, and 7 g of Na⁺MeO⁻ were added and stirred for an additional time. After 15 minutes, 8 ml of 1-fluoronaphthalene were added, the solution was heated to 110° C., and stirred for 26 hours.

To the reaction mixture was added water, followed by 10 ml HCl (5%) and 60 ml ethyl acetate. After phase separation, the organic phase was washed with brine, dried on MgSO₄, and concentrated to dryness to give 13.37 g of brownish oil containing 9.53% enantiomer R.

### Example 9

A 250 ml reactor equipped with a mechanical stirrer, and condenser was charged with 10 g of AT-OL and 60 ml DMA at room temperature under N₂. The mixture was stirred until complete dissolution, and 7.11 g of KOH were added and stirred for an additional time. After 30 minutes, 8 ml of 1-fluoronaphthalene were added, the solution was heated to 80° C., and stirred for 18 hours.

To the reaction mixture was added 90 ml of water, followed by 12 ml HCl (5%) and 60 ml ethyl acetate. After phase separation, the organic phase was concentrated to dryness to give 20 g of brownish oil containing 0.52% enantiomer R.

### Example 10

A 250 ml reactor equipped with a mechanical stirrer, and condenser was charged with 10 g of AT-OL and 60 ml DMA at room temperature. The mixture was stirred until complete dissolution, and 7.11 g of KOH were added and stirred for an additional time. After 30 minutes, 8 ml of 1-fluoronaphthalene were added, the solution was heated to 110° C., and stirred for 26 hours.

To the reaction mixture was added 90 ml of water, followed by 12 ml HCl (5%) and 60 ml ethyl acetate. After phase separation, the organic phase was concentrated to dryness to give 21 g of brownish oil containing 0.47% enantiomer R.

### Example 11

A 250 ml reactor equipped with a mechanical stirrer, and condenser was charged with 10 g of AT-OL and 60 ml DMA at room temperature under N₂. The mixture was stirred until complete dissolution, and 6 g of KOH were added and stirred for an additional time. After one hour, 8 ml of 1-fluoronaphthalene were added, the solution was heated to 80° C., and stirred at the same temperature. During the following 4 hours, two portions of KOH were added (6 g), and the reaction mixture kept at the same temperature for an additional hour.

To the reaction mixture was added water, followed by 12 ml HCl (5%) and 60 ml ethyl acetate. After phase separation, the organic phase was concentrated to dryness to give 25 g of brownish oil containing 4.85% enantiomer R.

Conversion of DNT to Duloxetine HCl

### Example 12

### Preparation of (S)-DNT-Base

A 2 liter reactor, equipped with a mechanical stirrer, was charged with a mixture of 100 g of (S)-(+)-DNT-Oxal, 600 ml of water, 96 ml of a 22 percent ammonium hydroxide solution, and 1 liter of toluene. The mixture was stirred at 25° C. for 20 to 30 minutes, and the organic phase was separated and washed three times with 300 ml of water, providing a toluene solution of (S)-DNT-base, which was used in Example 13 without evaporation.

### Example 13

### Preparation of (S)-duloxetine ethyl carbamate

A 1 liter reactor, equipped with a mechanical stirrer, thermometer, dean stark, and condenser, was charged with (S)-DNT-base obtained in Example 12 dissolved in 1020 ml of toluene and 13 g of K₂CO₃. The mixture was heated, and an azeotropic distillation of 284 ml of the mixture was performed. After cooling to 50° C., 47.46 ml of ethyl chloroformate were added over a period of a half hour, and the reaction mixture was stirred at the same temperature for an additional 2 hours. After cooling to room temperature, the reaction mixture was washed with 230 ml of water, 130 ml of a 5 percent HCl solution, 130 ml of water, 130 ml of a 5 percent NaHCO₃ solution, and 130 ml of water. The resulting toluene solution of (S)-duloxetine ethyl carbamate was used in Example 14 without evaporation.

### Example 14

### Preparation of (S)-duloxetine Base

A 1 liter reactor, equipped with mechanical stirrer, thermometer, and condenser, was charged with the solution of (S)-duloxetine ethyl carbamate in toluene prepared in Example. 13. The mixture was heated, and an azeotropic distillation of 268 ml was performed. After cooling to 60° C., 82.18 g of an 85 percent KOH solution were added and the mixture was heated to 94° C. for about 4 hours. After cooling to 60° C., 270 ml of water were added, and the resulting organic phase was washed three times with 270 ml of water, and treated with 4.6 g of charcoal (SX1) for 15 minutes, filtrated through a hyperflow bed, and washed with 60 ml of toluene. The solution was distillated at 30° to 40° C. under a vacuum of 20 to 30 mmHg until a volume of about 1 to 2 volumes of toluene was obtained. The resulting toluene solution of (S)-duloxetine base was used in Example 15.

### Example 15

### Preparation of (S)-(+)-duloxetine hydrochloric

A 1 liter reactor, equipped with mechanical stirrer, thermometer, and condenser, was charged with the solution of (S)-duloxetine-base in toluene prepared in Example 14. After cooling to room temperature, 670 ml of acetone were added, and the solution was heated to 30° C. Hydrogen chloride gas was bubbled into the solution until the pH the mixture was adjusted to 3 to 5, and the mixture was stirred at the same temperature for 1 hour. After cooling to room temperature, the resulting solid was filtrated out and washed three times with 100 ml of acetone. After drying in a vacuum oven at 45° C. for 15 hours, 47.5 g of (S)-(+)-duloxetine hydrochloride were obtained as an off white powder having a purity of 99.42%, based on HPLC area percent with an overall yield of 56.66%.

What is claimed is:

1. A process for preparing (S)-(+)-N,N-Dimethyl-3-(1-naphthalenyloxy)-3-(2-thienyl)propanamine (DNT), comprising combining S-(−)-N,N-Dimethyl-3-Hydroxy-3-(2-Thienyl)Propanamine (AT-OL) with a base selected from the group consisting of: alkali metal hydroxide, sodium metal alkoxides, lithium metal alkoxides, and a naphthalene selected from the group consisting of 1-fluoronaphthalene, 1-chloronaphthalene and mixtures thereof in a polar aprotic solvent selected from the group consisting of: C₅-C₈ aromatic hydrocarbons, ionic liquid, dimethyl Sulfoxide (DMSO), dimethylformamide (DMF), dimethylacetamide (DMA), acetonitrile, sulfolane, nitromethane, propylene carbonate and mixtures thereof to obtain DNT, wherein the reaction is conducted in the absence of a phase transfer catalyst.

2. The process of claim 1, wherein a solution of AT-OL in the solvent is combined with a base which combination is further combined with the naphthalene to obtain a reaction mixture.

3. The process of claim 2, wherein the mixture is heated to at a temperature of about room temperature to about the reflux temperature of the solvent.

4. The process of claim 3, wherein the mixture is at a temperature of about 35° C. to about the reflux temperature of the solvent

5. The process of claim 3, wherein after heating, the mixture is maintained, while stirring, for about 20 minutes to about 5 days.

6. The process of claim 1, wherein the base is an alkali metal hydroxide.

7. The process of claim 6, wherein the base is potassium hydroxide (KOH), or sodium hydroxide (NaOH).

8. The process of claim 1, wherein the base is a sodium metal alkoxides.

9. The process of claim 8, wherein the base is sodium methoxide.

10. The process of claim 1, wherein the base is added portion wise.

11. The process of claim 1, wherein the polar aprotic solvent is selected from the group consisting of toluene, xylene, dimethyl Sulfoxide (DMSO), dimethylformamide (DMF), dimethylacetamide (DMA), acetonitrile and mixtures thereof.

12. The process of claim 11, wherein the polar aprotic solvent is DMA or DMSO.

13. The process of claim 1, wherein the solvent is an ionic liquid.

14. The process of claim 13, wherein the ionic liquid is alkylammonium halides, alkylphosphonium halides, N-alkylpyridinium halides, N-N-dialkylimidazolium halides, tetraalkylammonium tetraalkylborides, 1-alkyl-3-methylimidazolium trifluoromethanesulfonate salts, monoalkylammonium nitrate salts, halogeoaluminate or chlorocuprate.

15. The process of claim 14, wherein the ionic liquid is 1-butyl-3-methylimidazolium tetrafluoroborate

16. The process of claim 1, wherein amount of R enantiomer of the DNT obtained is less than about 15% as area percentage HPLC.

17. The process of claim 16, wherein amount of R enantiomer of the DNT obtained is less than about 10% as area percentage HPLC.

18. The process of claim 17, wherein amount of R enantiomer of the DNT obtained is about 0.5% as area percentage HPLC.

19. The process of claim 1, further comprising the step of recovering DNT.

20. The process of claim 1, further comprising converting DNT to a salt.

21. The process of claim 20, wherein the salt is maleate.

22. A process for preparing duloxetine or a pharmaceutically acceptable salt thereof, comprising preparing DNT or salts thereof according to the process of claim 1 and converting the DNT to duloxetine or a pharmaceutically acceptable salt.

23. A process for preparing duloxetine or a pharmaceutically acceptable salt thereof, comprising reacting AT-OL with a base selected from the group consisting of: alkali metal hydroxide, sodium metal alkoxides, lithium metal alkoxides, and 1-fluoronaphthalene or 1-chloronaphthalene in a polar aprotic solvent selected from the group consisting of: C₅-C₈ aromatic hydrocarbons, ionic liquid, dimethyl Sulfoxide (DMSO), dimethylformamide (DMF), dimethylacetamide (DMA), hexamethylphosphoramide (HMPA), acetonitrile, sulfolane, nitromethane and propylene carbonate, wherein the reaction is conducted in the absence of a phase transfer catalyst and converting the DNT to duloxetine or a pharmaceutically acceptable salt thereof
